(19) United States
(12) Patent Application Publication
(10) Pub. No.: US 2023/0302048 A1
(43) Pub. Date: Sep. 28, 2023

(54) NOVEL METHODS FOR INCREASING HEALING SPEED OF HUMAN AND ANIMAL WOUNDS

(71) Applicant: Courtland Imel, Farmers Branch, TX (US)

(72) Inventor: Courtland Imel, Farmers Branch, TX (US)

(21) Appl. No.: 18/124,657

(22) Filed: Mar. 22, 2023

Related U.S. Application Data
(60) Provisional application No. 63/322,797, filed on Mar. 23, 2022.

(51) Int. Cl. A61K 33/30; A61P 17/02; A61K 33/06

(52) U.S. Cl. CPC A61K 33/30; A61P 17/02; A61K 33/06

(57) ABSTRACT

A composition of novel ions, and the use of the composition of novel ions in speeding up the healing process of mammal wounds with a reduction of scarring. In one exemplary embodiment these ions include Rubidium, Calcium, Zinc, and Potassium. These specific ions are referred to as a Protease Down Regulator (PDR).

### CLAIM OF PRIORITY

This application claims convention priority of U.S. Provisional Patent Application U.S. Ser. No. 63/322,797 filed Mar. 23, 2022, entitled Novel Methods for Increasing Healing Speed of Human Wounds, the teachings of which are incorporated herein by reference.

### TECHNICAL FIELD

This disclosure is directed to a composition for treatment of mammal wounds including in humans and animals.

### BRIEF DESCRIPTION OF FIGURES

The drawing FIGURES depict one or more implementations, by way of example only, not by way of limitations. In the FIGURES, like reference numerals refer to the same or similar elements.

FIG. 1 illustrates a method of treating a wound of a mammal.

### DETAILED DESCRIPTION

Reference now is made in detail to the examples illustrated in the accompanying drawings and discussed below.

The disclosure describes a composition of novel ions, and the use of the composition of novel ions in speeding up the healing process of mammal wounds with a reduction of scarring. Several inventive embodiments of the present disclosure are described below. In one exemplary embodiment these ions include Rubidium, Calcium, Zinc, and Potassium. These specific ions are referred to as a Protease Down Regulator (PDR).

Additional objects, advantages and novel features of the examples will be set forth in part in the description which follows, and in part will become apparent to those skilled in the art upon examination of the following and the accompanying drawings or may be learned by production or operation of the examples. The objects and advantages of the present subject matter may be realized and attained by means of the methodologies, instrumentalities and combinations particularly pointed out in the appended claims.

In the following detailed description, numerous specific details are set forth by way of examples in order to provide a thorough understanding of the relevant teachings. However, it should be apparent to those skilled in the art that the present teachings may be practiced without such details. In other instances, well-known methods, procedures, components, and circuitry have been described at a relatively high-level, without detail, in order to avoid unnecessarily obscuring aspects of the present teachings.

PDR is a wound management factor that normalizes aberrant biochemistry with non-healing wounds. Testing of PDR in cellular assays indicates that PDR acts on the gene expression level by down-regulating the genes for matrix metal proteinases and pro inflammatory cytokines, and up-regulates the genes for growth factors and tissue inhibitors of matrix metal proteinases. These four gene regulatory effects result in advantageous biochemical changes in the wound of human tissue toward a healing trajectory.

The present disclosure regulates the healing cascade at the gene expression level. The PDR ions regulate the ability to heal the wound faster by increasing epithelialization and granulation, while reducing edema, inflammation, and exudate accumulation. Through human studies on 11 patients, the use of PDR showed complete wound closure in 4 days, as compared to a hydrogel that closed the wound in 10 days. This process shows a 60% faster wound closure rate. The method involves a PDR that is a mixture of Rubidium, Calcium, Zinc, and Potassium. In a non-limiting exemplary embodiment, the mixture can be about 0.000001 to 10% Rubidium, 0.000001 to 10% Calcium, 0.000001 to 10% Zinc, and 0.000001 to 10% Potassium.

### Study

A study was performed on 11 human male patients with a mean age of 20.5. The study was a randomized, double blind, placebo controlled clinical trial. All subjects were healthy Caucasian males without a history of illness, infection, or pre-existing condition that may affect wound healing. The study involved 9 non-smokers and 2 smokers who reported smoking less than one pack per day.

Four similar wounds were placed on each subject: two wounds on the right thigh and two wounds on the left thigh. The wounds were placed, one proximal and one distal on each of the ventral aspects of the thighs. A computer program generated the type of treatment to be used on each wound site for each individual.

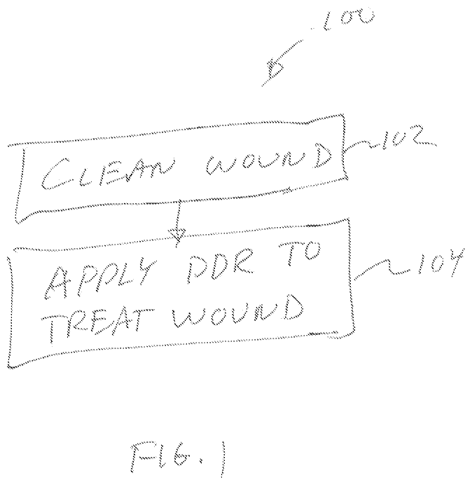

FIG. 1 illustrates method 100 having steps for treating a wound of a mammal.

At step 102, the wound is cleaned.

At step 104, the PDR is applied to the wound. The wound is covered with the PDR material and then covered with a bandage to allow healing to occur. The PDR can be embedded in the bandage.

Summary of Wound Re-Epithelialization

TABLE 1

| | Day After Wounding | | | | | | | | | |
|---|---|---|---|---|---|---|---|---|---|---|
| Treatment | 1 | 2 | 3 | 4 | 5 | 6 | 7 | 8 | 9 | 10 |
| Telfa | 0.00 | 0.00 | 0.00 | 0.00 | 0.36 | 0.09 | 0.45 | 0.73 | 0.82 | 0.73 |
| PEG Based | 0.00 | 0.00 | 0.18 | 0.09 | 0.64 | 0.27 | 0.36 | 0.64 | 0.55 | 1.00 |
| Placebo | | | | | | | | | | |
| Bencelok | 0.00 | 0.00 | 0.36 | 0.18 | 0.73 | 0.64 | 0.91 | 0.82 | 1.30 | 1.30 |
| Hydrogel | | | | | | | | | | |
| PDR Cream | 0.00 | 0.00 | 1.20 | 1.50 | 2.50 | 3.40 | 3.50 | 3.50 | 3.60 | 3.60 |

Measured Epithelialization on 11 patients. A value equal to 1.5 indicates wound closure and a value greater than 4 indicates complete wound healing. As shown in Table 1, the wound is over 50% closed after 5 days, and substantially closed by day 6.

Summary of Granulation Tissue Formation

TABLE 2

| | Day After Treatment | | | | | | | |
|---|---|---|---|---|---|---|---|---|
| Treatment | 3 | 4 | 5 | 6 | 7 | 8 | 9 | 10 |
| Telfa | 0.18 | 0.09 | 0.00 | 0.00 | 0.27 | 0.36 | 0.73 | 0.64 |
| PEG Based | 0.18 | 0.00 | 0.09 | 0.00 | 0.09 | 0.46 | 0.55 | 0.91 |
| Placebo | | | | | | | | |
| Bencelok | 0.36 | 0.09 | 0.18 | 0.46 | 0.55 | 0.55 | 0.91 | 0.91 |
| Hydrogel | | | | | | | | |
| PDR Cream | 0.82 | 1.00 | 1.00 | 1.00 | 1.00 | 1.30 | 0.82 | 1.00 |

Measured granulation tissue formation on 11 subjects. A value of 1.0 shows a closed wound. A value greater than 1 shows a mixture of scab with covering. As shown in Table 2, the use of PDR showed complete wound closure in 4 days, as compared to a hydrogel that closed the wound in 10 days. This process shows a 60% faster wound closure rate. The method involves a PDR that is a mixture of Rubidium, Calcium, Zinc, and Potassium.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as ±10% from the stated amount.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing has described what are considered to be the best mode and other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

1. A composition of ions configured to increase the speed of wound healing, wherein the composition of ions comprises a protease down regulator (PDR).

2. The composition of claim 1, wherein the composition comprises Rubidium, Calcium, Zinc, and Potassium.

3. The composition of claim 2, wherein the composition comprises 0.000001 to 10% Rubidium, 0.000001 to 10% Calcium, 0.000001 to 10% Zinc, and 0.000001 to 10% Potassium.

4. The composition of claim 2, wherein the PDR is configured to regulate an ability to heal the wound faster by increasing epithelialization and granulation.

5. The composition of claim 4, wherein the composition is configured to reduce edema, inflammation, and exudate accumulation.

6. The composition of claim 2, wherein the composition is suitable for topical administration on skin of a mammal.

7. A method of treating a wound of a mammal, comprising:
applying a composition to the wound, wherein the composition of ions comprises a protease down regulator (PDR).

8. The composition of claim 7, wherein the composition comprises Rubidium, Calcium, Zinc, and Potassium.

9. The composition of claim 8, wherein the composition comprises 0.000001 to 10% Rubidium, 0.000001 to 10% Calcium, 0.000001 to 10% Zinc, and 0.000001 to 10% Potassium.

10. The composition of claim 8, wherein the PDR regulates an ability to heal the wound faster by increasing epithelialization and granulation.

11. The composition of claim 10, wherein the composition reduces edema, inflammation, and exudate accumulation.

12. The composition of claim 8, wherein the composition is suitable for topical administration on skin of a mammal.
